(19) United States
(12) Patent Application Publication
(10) Pub. No.: US 2007/0129746 A1
(43) Pub. Date: Jun. 7, 2007

(54) Methods and devices for the treatment of neurological and physiological disorders

(76) Inventor: Hans Alois Mische, St. Cloud, MN (US)

Correspondence Address: HANS A. MISCHE, 32 HIGHBANKS PLACE, ST. CLOUD, MN, 56301, US

(21) Appl. No.: 11/504,514

(22) Filed: Aug. 14, 2006

Related U.S. Application Data
(63) Continuation-in-part of application No. 10/843,828, filed on May 11, 2004.
(63) Continuation-in-part of application No. 09/457,971, filed on Dec. 9, 1999, now Pat. No. 6,375,666.
(63) Continuation-in-part of application No. 10/056,323, filed on Jan. 24, 2002, now Pat. No. 6,764,498.
(60) Provisional application No. 60/727,446, filed on Oct. 17, 2005.

(51) Int. Cl. A61M 29/00

(52) U.S. Cl. 606/191

(57) ABSTRACT

Methods and devices are disclosed that provide treatment for neurological and physiologic conditions by affecting electrical, sensory, biochemical and biologic signal propagation.

### CROSS REFERENCE TO RELATED APPLICATION

This application is a continuation-in-part of application Ser. No. 09/457,971 filed December 9, 1999, now U.S. Pat. No. 6,375,666; application Ser. No. 10/056,323 filed Jan. 24, 2002, now U.S. Pat. No. 6,764,498, and application Ser. No. 10/843,828 filed on May 11, 2004. In addition, this application claims the benefit of provisional application with Ser. No. 60/727,446, and provisional application with Ser. No.________ which was filed on Aug. 15, 2005 and with whose application is included in this utility application for the record.

### FIELD OF INVENTION

The present invention relates generally to the modification of electrical conduction properties within the body. The device and methods are disclosed in the context of treating neurological and physiological disorders that affect a variety of anatomical organs and tissues.

### BACKGROUND OF THE INVENTION

The current methods of treating a range of neurological and physiological disorders include the use of systemic drugs, surgical procedures, tissue ablation, electrical stimulation and gene treatments. Many of these disorders are manifested by gross conduction defects. These neurological disorders are may affect many types of anatomical organs and tissues such as brain, heart, muscle, nerves and organ tissues.

### SUMMARY

In contrast to the prior art, the present invention proposes treatment of neurological disorders by subjecting selected tissues to localized mechanical stress. It is difficult to quantify the level of stress applied to the tissue; operable values will vary from low levels to high levels dependent on the type and location of tissue to be treated. The tissues treated can be of many types within the body such as the brain, heart, muscles, nerves or organs.

The invention is disclosed in the context of neurological disorders but other the inventive technology can also be used to treat a wide variety of organs and anatomical tissues, and the treatments of other types of ailments are contemplated as well. For example, other applications of this invention include placement in the pituitary, thyroid, and adrenal glands or in a variety of organs. In addition, placement of the inventive device in tumors may suppress growth due to nerve and vascular compression. The later may prevent blood-born metastasis to other parts of the body. Likewise, hemorrhaging can be stopped or reduced by vascular compression using the invention. Pain management in all parts of the body can be achieved by placement of the inventive device adjacent to selected nerves. Positioning an inventive stress-inducing device within the bone can accelerate healing of broken bones. Disclosure of this invention for neurological and neuromuscular applications is intended to be illustrative and not limiting.

In the treatment of treating cardiac arrhythmias, sometimes the result of a neuromuscular disorder, the inventive device can be positioned within, on, through, or adjacent to heart tissue in order to affect or block electrical conductions that cause symptoms such as atrial fibrillation, pacing defects, hypotension and hypertension. The inventive devices and methods can replace the current practice of RF ablation, surgical procedures (such as the Maze procedure) and anti-arrhythmia drugs. Proper shape, geometry, and placement of the devices can result in treating the tissue in a similar shape and fashion as those in the aforementioned treatments. The shape of the treatment of the typical Maze procedure can be replicated with the proper physical shape and placement of the inventive device. One embodiment of a device for a method of treating cardiac arrhythmia is a device similar to a rivet. The first end of the rivet would pass through the desired location of the myocardium and be positioned or seated on the external or internal surface, depending on approach. The second end of the rivet combination would be slid along the shaft of the rivet and seated on the opposite side of the myocardium as the first end of the rivet. The first and second end would then be advance towards each other resulting in compression, elongation or mechanical stressing of the myocardial tissue between and proximate to the rivet. The amount of mechanical stressing would be controlled by the distance form the first end to the second end.

The inventive devices and methods can be used in the treatment of cardiomyopathy. A primary cause of cardiomyopathy is a lack of the proteins dystrophin and collagen, the same protein deficiency that exists in the skeletal muscles and leads to generalized weakness, wasting and respiratory complications. Dystrophin and collagen is also needed by cardiac muscle, and its lack can lead to the loss of cardiac muscle cells under the stress of constant contraction. It is know that mechanical forces on tissues can generate increased deposition of collagen fibers within muscular tissues and strengthen these tissues. In the treatment of cardiomyopathy, the inventive devices can provide methods of selectively, broadly or focally, generating mechanical stresses that result in the therapeutic deposition or increased formation of collagen fibers. These fibers can then strengthen myocardial tissues muscle and retard or reversing the effects of cardiomyopathy. This phenomenon can also be used to treat other diseases and illnesses that affect tissue strength and connective tissue orientation, density and volume. In addition, the deposition or formation of collagen in a predetermined formation or matrix can allow of nerve growth along the collagen fibers. This can be useful in forming circuitry for heart conduction pathways as well as the growth of new nerves to treat spinal injuries or paralysis.

Many neurological disorders are a result of improper conduction of electrical currents in various brain tissues. In the case of Parkinson's disease, the conduction currents in the thalamus tissues become disorganized and cause conditions associated with the disease. Likewise, in epilepsy errant currents cause various levels of seizures. In cases of dystonia, errant currents originate in the basal ganglia. Depression and schizophrenia are associated with various electrochemical defects in other portions of the brain. Also, pain symptoms such as trigeminal neuralgia are associated with multiple sclerosis. Paralysis is normally a condition that results from brain injury, nerve damage, or nerve severing.

The localized stresses generated by the inventive device called a Mechanical Stress Device (MSD), will control, inhibit and direct current conduction by reorienting and/or reorganizing the electrical bias of the neurological tissues. In addition, applications for the MSD include compression of selected nerves in order to control, mediate, or suppress conduction along the nerve fibers and bundles that are associated with certain neurologic disorders. The localized stresses also can affect activate or suppress baroreceptors within arteries, veins, heart tissue and other tissues and organs. Affecting the baroreceptors can allow control of various physiologic functions such as sinus rhythm, sympathetic nervous system, blood pressure, hormonal activity and metabolism as examples. The inventive devices and methods can affect the wall of the carotid sinus, a structure at the bifurcation of the common carotid arteries. This tissue contains stretch receptors that are sensitive to mechanical and electrical forces. These receptors send signals via the carotid sinus nerve to the brain, which in turn regulates the cardiovascular system to maintain normal blood pressure. The proper method of use and placement of the inventive device can manipulate the baroreceptors and achieve regulation of the cardiovascular system in order to control blood pressure levels. For example, when place proximate to the carotid sinus, the MSD will apply localized stresses that modify or modulate the stretch baroreceptors. The MSD can be complemented with electrical properties and features that can provide additional affects to the baroreceptors function.

The MSD can be placed internal or external to arteries and veins in order to achieve desired activation of baroreceptors. MSD can be attached to external body plane; skin.

The MSD can also be utilized as an electrically conductive device that creates an electrical connection or “bridge” between targeted anatomical tissues. This technique may facilitate tissue-to-tissue communication, aid in regenerating nerve connections, or affect the electrical conduction between the SA and AV nodes of the heart to overcome pacing defects. Likewise, an MSD may be placed proximate to the pulmonary vein in order to quell, block or mitigate abhorrent conduction currents that cause atrial fibrillation.

In the case of Parkinson's disease, an MSD is implanted in the tissues proximate to the thalamus and induce localized stresses that cause depolarization of the thalamus tissue and thus eliminate or reduce the symptoms of the disease. In Dystonia, the MSD is positioned proximately to the basal ganglia and disrupts the electrical disturbances associated with this disorder.

The same effect is utilized in the treatment of epilepsy and other tissues when the MSD is installed in the targeted brain tissues. An MSD may be place on or adjacent to the vagus nerve in order to mechanically and or electrically cause stimulation. This stimulation of the vagus nerve can provide therapeutic treatment of epilepsy and depression. In addition, MSD stimulation of the vagal nerve can provide treatment for heart function such as cardiac ventricular output, rhythm, and systemic blood pressure. The devices and methods associated with the MSD can also be utilized in the sinuses and various ventricles of the brain to treat personality disorders such as schizophrenia or depression. Additionally, migraine headaches and Tourette's Syndrome may be treated with the MSD technology. In general, the methods of the invention guide the placement of the device to ensure a therapeutic effect from the device. In another application, Vestibular disorders, which may interact with blood pressure and heart rate control, can be treated and controlled. The vestibular system is one source of information about uprightness and the system has an affect on the cardiovascular system. Proper placement and manipulation of the vestibular nerve with one or more of the MSD design embodiments can alleviate or control heart rate and blood pressure, as well as physical balance.

The MSD technology may also be used to affect the neurologic response of the digestive system in order to control appetite, digestion or metabolism. In addition, using the previously invented methods and devices in this and the cross referenced patent and applications by Mische, the MSD technology can be used to treat urge or stress incontinence by affecting nerve conduction and neuromuscular function. Also, the neurological and neuromuscular function of the reproductive system can be treated and controlled by using the MSD technology to modify transport and expression of hormones, sperm, ovum, and fluids.

The MSD can be permanently implanted or used acutely and then removed. Likewise, the device can be fabricated of biodegradable materials that are placed chronically and allowed to biodegrade over time.

The devices and methods can be used alone of in conjunction with other therapies.

Examples of electrical therapy with various MSD embodiments are given and they include pacing, depolarization, ablation, and tissue alteration.

MSD devices can be configured so that they deliver treatment on a temporary basis and are then removed or disabled. For example, a device such as in FIG. 7 could be used for a temporary treatment regimen or method. The device would be deployed, positioned, expanded for a period of time and then retracted and removed when desired. It could also be used in conjunction with an electrical stimulator. In another embodiment, the device could be a balloon construction that is inflated for the treatment period and then deflated and removed. Additionally, the balloon construction could also have one or an array of electrodes on the surface, as well be made of electrically conductive polymers. The treatment regimen would cease when desired, or if undesired clinical results are observed. The long term result could be attained when the tissues which caused the negative illness state were “retrained” by the MSD type device and further treatment would not be necessary. Additionally, physical remodeling of the tissue may be the result of a temporary treatment regimen. In some therapeutic cases it may be beneficial to treat in a method that allows the MSD to be placed at the treatment site and the delivery system is left engaged for a period of time. This period of time could be used to observe, measure the effectiveness of the treatment and/or allow a modification to the treatment parameters during this period of time.

MSD devices can be configured so that upon delivery to the desired location within the tissue or body, they are detached from the delivery device by unscrewing, detent release, release of compression or adhesive, or release of other means of securing the MSD to the delivery device. Other means of securing the MSD to the delivery device includes forceps, graspers, swaging, jamming, wedging, friction, tying, magnetics, electrical discharge, melting, fusing, defusing, grapples, etc.

MSD devices can be configured so as to release a therapeutic substance or drug when activated by external or in situ mechanical, chemical or electrical stimuli. These stimuli can actually be provided and distributed by the treated/malfunctioning tissues or tissues proximate to the treated/malfunctioning tissue. The stimuli can be provided by the tissue from localized spasms originating from tissue, muscle or organs, as well as abhorrent electrical signals or biochemical release generated by the diseased/affected tissues. Delivery of the therapeutic substances could continue until the tissues are inactivated and associated symptoms are thus relieved.

In some clinical cases, it may be necessary to contract a volume of tissues. Instead of a device being therapeutic in its expansive state, it may also provide therapy during volumetric contraction. One example could be a device, similar to FIG. 7, with grapples or hooks that are placed within a brain ventricle. Upon activation, the device could grab the walls of the ventricle and collapse or contract volumetrically. Another embodiment would be a device that is expanded in order to grasp tissue and then retracted to contract, elongate or stretch the tissue in a predetermined direction and stress strain parameters. This invention could be used in other types of ventricles, cavities or openings. This can also be used in solid tissues, bones, and organs. MSD technology can be used to expand ventricles and ducts within brain tissues and organs so as to improve drainage of fluids, relief of tissue-to-tissue interface, and to relieve or improve physiologic pressures within a ventricle, or between a ventricle or duct. For example, an expandable MSD can provide a device technology and a treatment method for opening brain ducts and draining excess CSF from the brain.

MSD's can provide a form of mechanical dilatation of tissue. Means of creating tissue dilatation include dilator tools that are on a shaft with the treatment end having physical features that can be one or more of the following: diametrically tapered, rounded, blunt, inverted, or expansive.

MSD's can provide a substrate for carrying neurons or other biologic compositions. These types of devices can also be used to treat many other types of neurologic or physiologic disorders.

MSD's can use their inherent geometries to prevent migration after placement at he treatment site. Additionally, complementary features can be incorporated to the device so that they do not migrate after placement. These complementary features can include spikes, hooks, sutures, bumps, voids, threads, barbs, inverted wedges, filaments, coarse surfaces, adhesives, etc.

MSD's can be constructed so as to be affected by the change in temperature of the tissues proximate to the treatment sites. In some cases, these temperatures may be a result of abhorrent electrical signals, chemical response or mechanical forces within the tissues proximate of the treatment site. When the temperature changes, the physical properties of the MSD changes, as well as the affects of to the brain (i.e., localized stresses and strains). This can be accomplished by the use of temperature sensitive materials such as Nitinol or bimetallic structures. Other embodiments may use polymers and metals which change shape when affected by electrical, chemical, light, or mechanical energy.

An MSD can be controlled utilizing thermally, pneumatically, or with magnetostrictive properties of the construct.

An expandable preformed MSD can be shaped appropriately (i.e., trapezoid, rectangular, tubular, conical, curved, etc) in order to bias the therapeutic stresses to tissues and avoid imparting stressed to tissues. A MSD's expansion can be controlled by magnetic coupling to ajack, screw or ratcheting mechanism. An external magnet outside of the body would be manipulated to cause an interaction with the implanted MSD. The external magnet may spin and, via coupling, cause a screw to turn and effect the sizing of the MSD, modifying the stresses imparted to the tissue. Likewise, a miniature motor assembly in the MSD can be used to drive the expansion or contraction of the MSD. The expansion and contraction can be modulated one-time, many times over a period, or at a repetitive frequency that causes sustained or short term vibrations. The motor can be operated by an implantable battery system, utilize a hardwire connection to a generator, coupled inductively or capacitively, or magnetically

It has been shown that stress to tissues can result in localized increase of collagen deposits. These collagen deposits can improve tissue strength as well as create a matrix for nerve regeneration. The orientation of stresses created by the MSD devices can predetermine the deposition of collagen and nerves. This phenomena can be use to reconnect severed nerves or reroute nerves and electrical conduction pathways within tissues such as the brain and heart.

MSD can physically, biologically, mechanically, chemically or electrically modify production of detrimental biochemical/brain chemistry such as dynorphin or a chemical in the brain called CREB or cyclic AMP responsive element binding protein, which can cause depression, anxiety or other maladies. Biological and chemical additives to the MSD can scavenge or modify detrimental biochemical/brain chemistry such as dynorphin that can cause depression or other maladies. Likewise, MSD's can modify the action potential of the brain cellular make-up by reversal of the electrical potential in the plasma membrane of a neuron that occurs when a nerve cell is stimulated; by changing the membrane permeability to sodium and potassium.

MSD technology in the form of a balloon can provide a number of design alternatives and treatment methodologies. For example, a balloon that conforms to the cortical surface of the brain can provided constant or variable localized stresses that provide therapy. The balloon surface could be smooth or flat, or could have projections or bumps that contact the brain tissue in a predetermined fashion. This allows for distinct and focal stresses and strains on brain tissue. The MSD balloon can be controlled by the connection to an implantable pump mechanism. The pump regulates the expansion and deflation of the balloon in order to customize the size and shape of the balloon. This allows for varying levels of stress to the tissue. The pump can be controlled by a wireless remote control via the likes of inductive coupling, RF or Digital communications, etc. Also, the pump could be controlled by hardwire connection to a control module. The pump could be controlled by health care personnel or by the patient. A balloon can be shaped appropriately (i.e., trapezoid, rectangular, tubular, conical, curved, etc) in order to bias the therapeutic stresses to tissues and avoid imparting stressed to tissues.

An MSD can be placed anywhere in the body so that it impacts neurologic tissues and provides therapy. These areas include Area 25 in the brain to aid in treating depression. A MSD can be placed proximate the pudenal nerve to treat incontinence.

All MSD designs can be positioned within tissues in a remote location from the region where an abhorrent signal is originating. In this case, the MSD can interrupt a signal pathway, circuit, or transmission line. For example, an MSD can be placed on the cortical surface of the brain. Placement and stress applied in the proper location can treat/control a number of physiologic functions (i.e., atrial fibrillation, pain, incontinence, blood pressure, hormonal activity, etc). For example, proper placement on the cortical surface can help treat Parkinson's tremors by interrupting or modifying the corto-basal ganglia motor control loop. An MSD may be formed in a Cartesian coordinate fashion so as to be able to program the affect to the tissue in the most desirable fashion.

A MSD can be positioned on the spinal column, spinal nerve or vertebral nerves to block or dissipate abhorrent signals and/or pain in remote regions of the body.

An MSD can be used to treat sciatica by placement directly on the sciatic nerve or in the spinal column nerve bundle. Likewise, scoliosis may be treated by selective treatment of nerves and/or nerve bundles with a MED. Additionally, an MSD can provide treatment of atrial fibrillation by placing a MSD in the proper location of the spinal nerve/bundle column to control the fibrillations

A MSD in the form of an expandable Deep Brain Stimulator (DBS) lead can be used to apply controlled stresses, record EEG and other parameters, connect to generator for stimulus. These actions can be done simultaneously, sequentially, or in an order determined by the operator. A MSD in the form of a DBS lead can be used temporarily or implanted permanently. The expansion of the MSD at the tip of the lead can be controlled at the proximal end of the lead by pulling, pushing, twisting, sliding, and mechanisms. The sizing of a MSD can be controlled by power or information provided by a DBS or electrical generator. A separate “communication” channel can be used to send signals or power to the MSD that dictate the expansion, contraction or vibration of the MSD. The generator/MSD configuration would thus provide the ability to treat the patient with complementary effects. An MSD can be configured in such a fashion so as to accept or “dock” with a standard DBS lead. Likewise, it can be configured so as be disengaged or “undocked”.

MSD's that pinch neurologic tissues (brain, connective tissues, nerves, muscles, organs, etc) alter the electrical and/or chemical properties. This phenomenon is useful in treating disorders. MSD's can be implanted that electrically or chemically neutralize tissue to treat disorders. The tissues electrical potential can be “grounded” to dissipate the abhorrent signals. The tissues chemical potential can be changed by affecting the pH of certain regions by inserting chemicals, drugs, or elements that modify these regions pH. These substances can be inserted alone or be part of a complex treatment regimen or on a device (permanent implant or temporary implant). An MSD device can also be useful in suppressing or deterring the formation of lesions associated with multiple sclerosis.

The MSD can be a partial or complete band or hoop that goes on or around a portion of the brain or the entire circumference. The MSD may be activated manually through the skull by having a portion of the MSD protruding from the skull bone that is manually activated by the patient or medical personnel. The manually activated portion can be under the scalp or protrude from the scalp.

A MSD can be used to treat ulcer (stomach, intestinal, diabetic, etc) when placed proximate an ulcer and cutting off blood flow and neurologic activity. The MSD can be placed endoscopically or angiographically if needed.

An MSD electrode can be made with moveable sheath that allows controlled exposure of one or more electrode elements as necessary. Exposure may occur by the projection or expansion of the electrode element(s) in a radially, axial, or longitudinal fashion. Electrode construction with multiple barbs that project in a racially and/or longitudinal or axial direction. If desired, barbs/projections can be electrically and mechanically independent from each other. An MSD electrode with a moveable sheath will provide variable exposure and expansion of electrode element. An MSD electrode can be made so that the operable portion is biased in a predetermined radial direction from the axis. The radial direction can encompass from 0 to 360 degrees. In alternative embodiments, there may be a number of elements that are independently controllable in order to customize the electrodes projections and effects.

### BRIEF DESCRIPTION OF THE DRAWINGS

Throughout the several views of the drawings several illustrative embodiments of the invention are disclosed. It should be understood that various modifications of the embodiments might be made without departing from the scope of the invention.

Throughout the views identical reference numerals depict equivalent structure wherein:

FIG. 1. is a schematic diagram of the head showing mechanical stress devices implanted within brain tissue.

FIG. 2. is a schematic diagram of the head showing mechanical stress devices implanted in the frontal sinus, lateral ventricle of brain, and between the skull and brain tissue;

FIG. 3. is a schematic diagram of the head showing the mechanical stress device delivery system;

FIG. 4. is a schematic diagram of the head showing the mechanical stress device delivery system;

FIG. 5. is a schematic diagram of the head showing the mechanical stress device delivery system;

FIG. 6. shows a variety of MSD designs, and

FIG. 7. depicts an MSD, which is manually expanded contracted.

### DETAILED DESCRIPTION

The device and methods, which are similar to those discussed in the patent application filed on Nov. 19, 1999 by Mische entitled, “Mechanical Devices for the Treatment of Arrhythmias” which is incorporated by reference herein.

Throughout the description the term mechanical stress device MSD refers to a device that alters the electrical properties or chemical properties of physiologic tissues. The device may be made of metal such as Nitinol or Elgiloy and it may form an electrode for electrical stimulation. One or more electrodes may be associated with it. The MSD may incorporate fiber optics for therapeutic and diagnostic purposes. The device may also be made from a plastic or other non-metallic material. The MSD may also incorporate a covering of polymer or other materials. The MSD may also be a composition of different materials. The MSD may be smooth or have cutting or abrasive surfaces. The MSD may have, but not limited to, other elements that protrude from the contour of the surfaces such as spindles, splines, ribs, points, hooks, wires, needles, strings, and rivets.

The MSD may be implanted for chronic use or for acute use. Biodegradable materials that degrade or dissolve over time may be used to form the MSD. Various coatings may be applied to the MSD including, but not limited to, thrombo-resistant materials, electrically conductive, non-conductive, thermo-luminescent, heparin, radioactive, or biocompatible coatings. Drugs, chemicals, and biologics such as morphine, dopamine, aspirin, lithium, Prozac, genetic materials, and growth factors can be applied to the MSD in order to facilitate treatment. Other types of additives can be applied as required for specific treatments.

Electrically conductive MSDs, or MSDs with electrode elements, may be used with companion pulse generators to deliver stimulation energy to the tissues. This electrical therapy may be used alone or in combination with other therapies to treat the various disorders. Electrical therapies may be supplied from implantable devices or they may be coupled directly to external generators. Coupling between the MSD and external generators can be achieved using technologies such as inductive, capacitive or microwave coupling as examples. The MSD may also be designed of geometries or materials that emit or absorb radioactive energies.

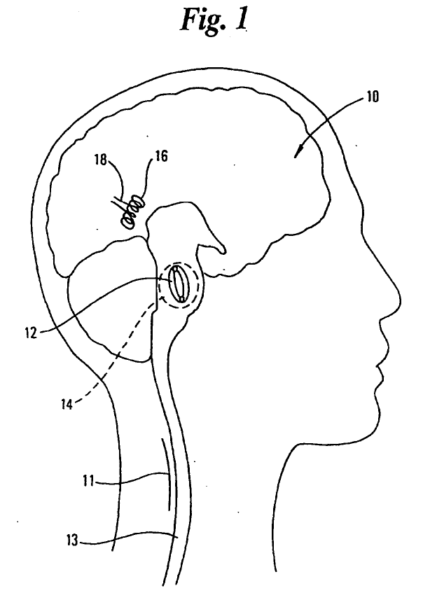

FIG. 1 is a schematic diagram showing several possible locations and geometries for the mechanical stress device (MSD) within the brain 10. A multi-element splined MSD 12 is positioned proximate to the thalamus 14. In this case, the treatment is for Parkinson's disease. A coil MSD 16 is positioned proximate to the trigeminal nerve 18 for treatment of trigeminal neuralgia. A wire form MSD 11 is positioned adjacent to the spinal cord 13.

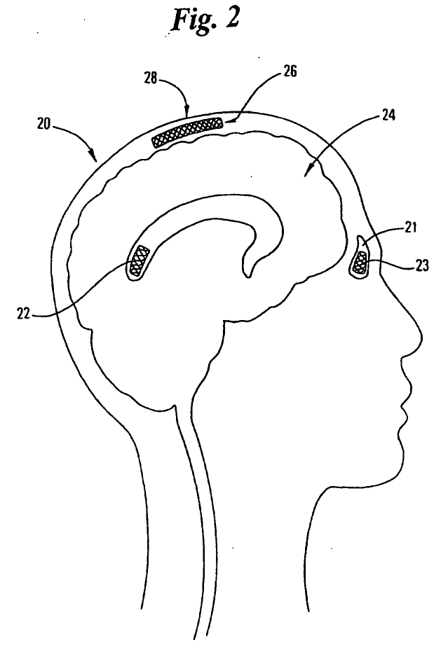

FIG. 2. is a schematic diagram of the head showing 20 various locations of MSDs of a tubular mesh form. An MSD 22 is located in the lateral ventricle of the brain 24. Another MSD 26 is positioned between the skull 28 and the brain 24. Within the frontal sinus 21 an MSD 23 is positioned.

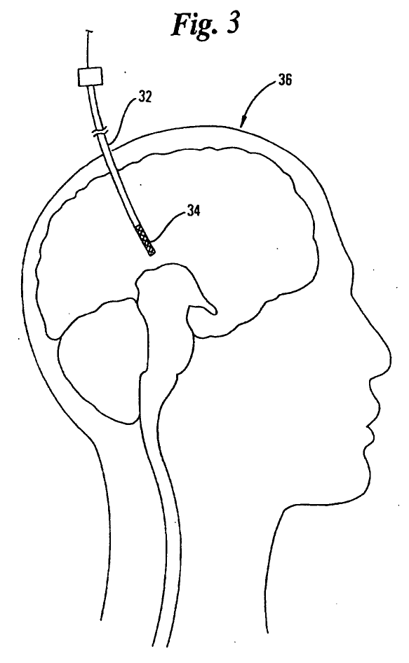

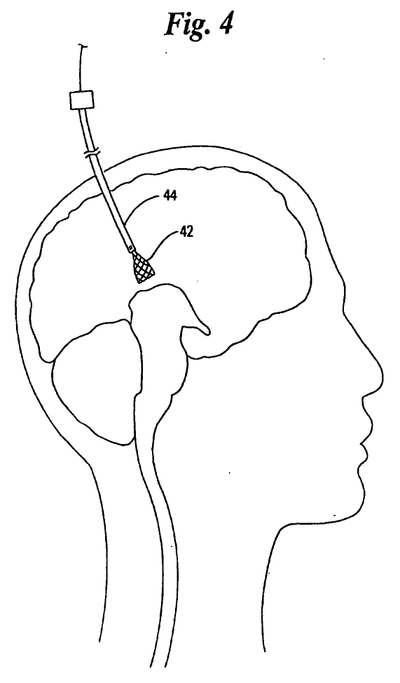

FIG. 3 and FIG. 4 should be considered together. Together the two figures show the deployment of an MSD.

FIG. 3 is a schematic diagram of a tubular mesh type MSD delivery system. The tubular catheter 32 delivers the tubular mesh MSD 34. The first stage of implantation is navigation of the device to the selected site through the skull 36.

FIG. 4 shows the tubular mesh 42 expanding into position as it emerges from the lumen of the delivery catheter 44. In the self-expanding case, the tubular mesh has a predetermined maximum expandable diameter. The mesh can be made of a shape-memory material such as Nitinol so that when subjected to body temperature the structure expands. With shape memory materials, the shape of the expanded device can be predetermined. Additionally, the device can be retrieved, repositioned, or removed by using its shape memory characteristics. In general the MSD may be used acutely or chronically depending on the disease state of the patient.

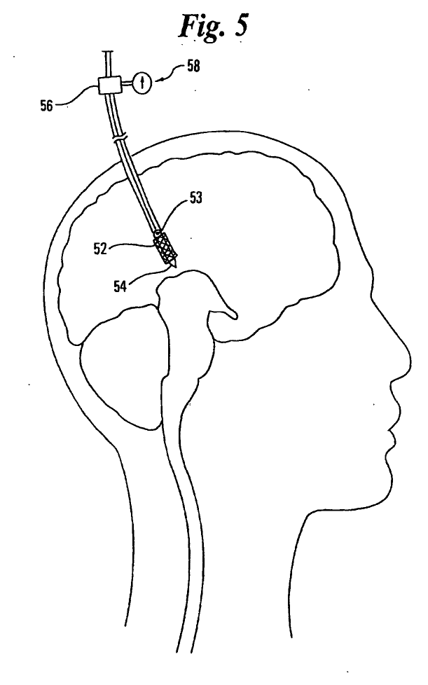

FIG. 5 shows an alternate balloon expanded MSD 52. In this alternate embodiment a balloon 54 may be used to expand the device within or proximate to selected tissues. In the balloon expandable case, the balloon may have a predetermined minimum or maximum diameter. In addition, the balloon shape can be made to provide proper placement and conformance of the device based on anatomical requirements and location. The balloon may be covered with electrically conductive material. The balloon may be inflated via a syringe 56 and a pressure gauge 58. For example an electrode site 53 may be connected to a remote pulse generator (not shown) to stimulate or ablate the site. The stimulator may activate the electrode either chronically or acutely.

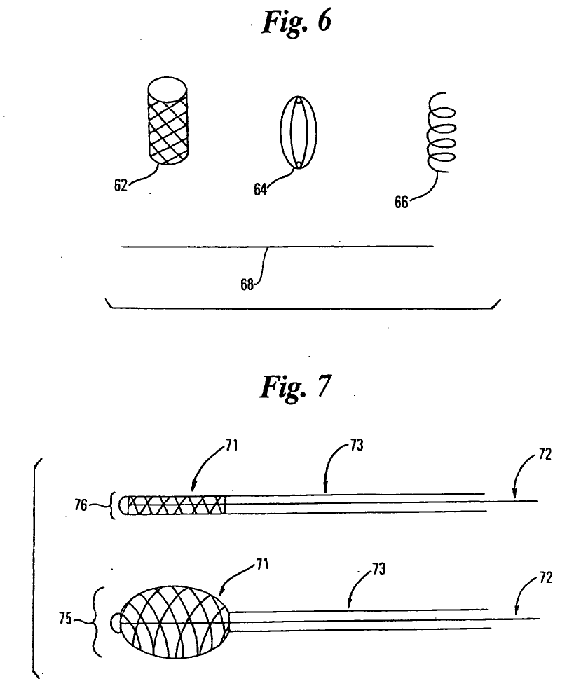

FIG. 6 shows a variety of possible MSD shapes and geometries. A tubular mesh 62, a multi-element spline 64, a coil 66, a wire 68 are all acceptable shapes for the MSD although each shape may be specifically adapted to a particular disease state. Other anticipated geometries include clam shells, spherical shapes, conical shapes, screws, and rivets. Although the preferred embodiments consider expandable geometries, alternate geometries can be constructed that retract, compress, collapse, crimp, contract, pinch, squeeze or elongate biologic and physiologic tissues as long as they provide one or more of the desired mechanical, electrical or chemical effects on the selected tissue. Delivery methods for the different possible geometries are anticipated, too.

FIG. 7 shows two states of a manually expandable MSD device 71. The device consists of a coaxial shaft 72 and tube 73 arrangement. Attached to the distal end of the shaft 72 and the tube 73 is a braided mesh tube MSD 71. When the shaft 72 and tube 73 are moved opposite of the other by manipulating the proximal ends, the MSD 71 expands 75 or contracts 76. In this case, the MSD 71 can be made of any structure that expands and contracts such as a coil, splined-elements, etc. The various methods of expanding and contracting these structures are, but not limited to, push-pull, rotation, and balloon manipulation. In this type of device, direct connection to either an electrical generator, laser, or monitoring system can be made. In addition, it be envisioned that a device of similar nature be connected to a mechanical energy source, such as rotational or vibrational, in order to increase localized stresses.

The MSD can also utilize devices such as a balloon catheter, expanding devices, or wedges that impart stress or certain levels of localized trauma to selected tissues. The resultant stress and trauma affect the tissues so that current conduction in modified. It is envisioned that any of these devices can be used alone or in conjunction with other treatment modalities in order to provide the desired therapeutic result.

In general, the MSD will have a relaxed or minimum energy state. However the device or the implantation procedure should stretch or stress the device so that it applies a persistent force to the tissues to alter conduction in the strained tissues. In this sense the implanted MSD is not in a fully relaxed state after implantation. In some instances the MSD will cause the tissues to yield or tear generating altered conduction.

Preferably, the MSD is delivered in a minimally invasive procedure such via a catheter or other device. X-ray imaging, fluoroscopy, MRI, CAT scan or other visualization means can be incorporated into the procedural method. In general the devices maybe introduced with cannulas, catheters or over guidewires through naturally occurring body lumens or surgically prepared entry sites. It should be apparent that other surgical and non-surgical techniques can be used to place the devices in the target tissue.

It should be apparent that various modifications might be made to the devices and methods by one of ordinary skill in the art, without departing from the scope or spirit of the invention.

In another embodiment, MSD's may also be designed in order to optimize coupling with external sources of electromagnetic energies via inductive or capacitive coupling. These energies can be utilized to electrically activate the MSD in order to impart voltages and currents to tissues to augment the mechanoelectric and or mechanochemical effects of the MSD. The MSD can be designed in such a fashion where it acts similarly to an implanted antenna. Likewise, the MSD may function primarily as an antenna with little, if any, mechanoelectric effects. The coupled electrical energy to this MSD antenna can be directly imparted to the tissues adjacent to the implanted. The received energy may be used to charge a circuit that is integrated into the MSD structure that discharges at a certain level, directing electrical energy to the desired or adjacent tissue. For example, the circuit may consist of resistors, capacitors, inductors, amplifiers, diodes or other components that assist in producing the desired function and effects. The circuit may consist of separate nodes for input and output voltages or it may have one node for both input and output. The MSD may also have a discrete antenna or antenna-circuitry for receiving or transmitting energy and/or information.

In another embodiment, the MSD may consist of circuitry that can automatically treat the neurological defects by utilizing the electrical energy generated by the physiologic tissues in which the MSD is implanted. In the case of epilepsy, focal tissues generate errant currents that result in seizure activity. These affected focal tissues are readily identified with standard CAT or MRI imaging systems and an MSD can then be implanted into these tissues. When the errant currents are generated, these currents charge the circuitry in the MSD. When the circuitry is charged to a predetermined level, it discharges back into the affected focal tissues and resolves the errant currents. A RC time constant circuit can be utilized for this MSD version. Amplifiers, signal generators and other processing circuitry can be incorporated into an MSD in order to increase or modify the output.

In another embodiment, the MSD has a covering to increase the surface area of the device. The covering can encompass the entire device or selected portions and can be positioned on the outside or inside surface. Such a covering can be made of polymers such as Teflon, polyethylene, polyurethane, nylon, biodegradable materials or other polymeric materials. The covering can also be made of a fine metal or polymeric mesh. In all cases, the covering can be bonded to the surface of the MSD or applied as a loose sheath-type covering. The covering can have therapeutic materials applied or incorporated into the covering material itself Examples of the therapeutic materials include drugs, stem cells, heparin, biologic materials, biodegradable compounds, collagen, electrolytes, radiopaque compounds, radioactive compounds, radiation-activated substances, or other materials that enhance the clinical effects and/or procedures.

In another embodiment, the MSD may have a material that substantially fills its interior space. Such a material would prevent formation of spaces or voids once an expandable MSD is placed. The materials may be fibrous, gels, porous, foam or sponge-like and may be incorporated with polymers, glass, metals, radioactive compounds, biologic tissues, drugs, or other suitable materials that may enhance clinical effective and/or procedures. The materials would be flexible enough to allow expansion of the MSD and can be made of polymers, glass, metal, biologic tissues, drugs, or other suitable materials. Although not limited to, examples of biologic materials include stem cells, brain cells and matter, thalamic tissues, and collagen.

The use of appropriate materials may also provide certain electrical properties to the MSD that enable it to receive, store and/or transmit electrical energy. The dielectric properties of these materials would provide electrical capacitor properties and function to the MSD. This provides the benefit of creating an electrical circuit that can receive, store and discharge energy from various sources. The source may be external generators that couple capacitively, inductively or magnetically, RF energy from a predetermined portion of the electromagnetic spectrum to the MSD. In addition, the source may be an electrical generator connected by a wire or a cable to the MSD.

Another means of generating therapeutic electrical energy is to utilize galvanic effects. Proper material selection and interaction with physiologic fluids and tissues would result in galvanic currents or electrochemical reactions being generated by the MSD. Generally, dissimilar metals or materials would be used in order to optimize the generation of galvanic currents. These currents could provide constant therapeutic electrical energy levels to the desired tissues. This could potentially benefit patients suffering from Parkinson's, epilepsy, pain, depression, migraines, etc. The galvanic currents can also be used to energize, activate, or charge circuits or batteries that provide monitoring, diagnostic or therapeutic effects. This technology could also be used for intravascular devices such as stents in order to prevent thrombosis or hyperplasia or to energize implantable sensors or monitoring devices. Galvanic devices can also be used to treat peripheral pain, generate revascularization of myocardial tissues, treat tumors, provide electrical potential for drug transport into tissues, treat endometriosis, or to power, energize, activate, operate or charge other medical devices such as cardiac pacemakers, defibrillators or other electrical generator based systems.

In another embodiment, the MSD may be a structure that completely or partially slices into tissue. The slicing action cleaves or separates the tissue physically breaking the electrical conduction paths. In this case, the MSD can reach complete or partial state of expansion. In the case of complete expansion, the residual stress to the tissue would be approaching zero, while the partial expansion would result in a combined clinical effect via part mechanical stress and part slicing of tissue.

Additional methods of constructing MSD's include using three-dimensional structures such as wedges, slugs, clips, rivets, balls, screws, and other structures that impart stress to the tissues. Materials such as open-cell polymers, gels, liquids, adhesives, foams can also be inserted or injected into tissue and tissue spaces in order to generate the desired amount of stress. These types of material could also have the additional benefit of being therapeutic agents or carriers for therapeutic agents.

Another MSD structure can consist of a balloon that is positioned at desired location, inflated within the tissue, and then detached and left in an inflated state. Examples of inflation media can be fluids, gels, foams, pharmaceuticals, and curable resins.

Other embodiments of MSD composition include construction using magnet and magnetic materials that complement the localized effects of the MSD by controlling the electrical properties of the tissues using gradients and fields. In the case where the MSD is composed of magnet materials, the magnetic field emanating from the magnetic materials would bias electric fields within the tissues. This effect can control the direction of current conduction within the tissues. In the case where the MSD is composed of magnetic materials that interact with magnetic gradients and fields, an external magnet placed proximate to the head can physically manipulate the MSD. Movement of the magnetic would cause movement of the MSD. The manipulation would result in dynamic stresses to the tissues adjacent to the MSD, thus affecting the electrical properties of the tissues and potentially resolving seizures or tremors.

Other MSD can be built with an integrated circuit consisting of a resistor, capacitor, and an inductor. The inductor couples with the external electromagnetic energy and the resulting current generated in the inductor charges the capacitor. Based on the RC time constant of the circuit, the capacitor charges to a certain level and then discharges directly to the desired tissues and the errant currents are disrupted by this discharge. A combination of electromagnetic coupling and direct connection incorporates a generator with a transmission coil and a ground connection made directly to the patient, providing a closed-loop circuit. The ground connection can be made directly to the skin of the patient using a clip or a grounding pad such as used during electrosurgical procedures. The pad may be applied to the patient with tape, bands or adhesives. The ground connection may also be implanted on or within tissue. External generators may be manually operated by the patient or other person or may be automatically operated utilizing monitoring systems that identify seizures or tremors and energize the MSD. Likewise, automatic circuitry such as the aforementioned RC-timing circuit can be used. The generators may also be programmed to energize at a certain predetermined sequence, rate and level. In the treatment of mania, depression, schizophrenia or similar disorders, the generator may provide a constant output to maintain a consistent state of electrical condition of the tissues. For convenience, the external generators may be attached directly to the head or incorporated into a hat, helmet, or band. Alternately, the transmission coil separately may be attached directly to the head or incorporated into a hat, helmet, scarf or band. The coil may encompass the entire head or specific portions in order to attain desired coupling with the MSD. In addition, strain gauge technology can be incorporated that can measure and correlate the amount of mechanical stress and strain imparted to tissues or stress and strains imparted to the device by tissues and active organs such as vessels, hearts, valves, and other organs and tissues. Such data can be used to provide a feedback means by which to control the MSD in order to provide treatment as necessary based on the physiologic response or activation.

Likewise, as mentioned previously, the electrical energy inherent in physiologic tissue may also be the source that energizes the circuit. Again, it should be noted that various modifications might be made to the devices and methods by one of ordinary skill in the art, without departing from the scope of the invention.

What is claimed:

1. A method of treating depression comprising the steps of: identifying the target tissue responsible for depressive activity; placing a mechanical stress device proximate to the target tissue with a placement device; removing said placement device, whereby said mechanical stress device remains proximate to the target tissue; whereby said mechanical stress device affects the electrical activity conduction of the target tissue; whereby the affected electrical activity of the target tissues mitigates the depression.

2. A method as in claim 1, where the device is a permanent implant.

3. A method as in claim 1, where the device is a temporary, removable implant.

4. A method as in claim 1, where the device is directly coupled to an electrical generator.

5. A method as in claim 1, where the device is remotely coupled to an electrical generator.

6. A method as in claim 1, where the device is made of a magnetic material

7. A method as in claim 1, where the targeted neurologic tissue is the region in the brain known as cingulate area 25.

8. A method as in claim 1, where the targeted neurologic tissue is the region in the brain known as the cortical surface.

9. A method as in claim 1, where the targeted neurologic tissue is the region in the brain known as the prefrontal cortex.

10. A method of treating migraine headaches comprising the steps of: identifying the target tissue responsible for migraine activity; placing a mechanical stress device proximate to the target tissue with a placement device; removing said placement device, whereby said mechanical stress device remains proximate to the target tissue; whereby said mechanical stress device affects the electrical activity conduction of the target tissue; whereby the affected electrical activity of the target tissues mitigates the migraine headache.

11. A method as in claim 10, where the device is directly coupled to an electrical generator.

12. A method as in claim 10, where the device is remotely coupled to an electrical generator.

13. A method of treating neurologic disorders comprising the steps of: identifying the target tissue responsible for the disorder; placing a mechanical stress device proximate to the brain's cortical surface; the whereby said mechanical stress device remains proximate to the cortical surface; whereby said mechanical stress device affects the electrical activity of the appropriate cortical-sensory pathway connecting the cortical surface and the target tissue; whereby the affected electrical activity of the cortical-sensory pathway mitigates the neurologic disorder.

14. A method as in claim 13, where the device is directly coupled to an electrical generator.

15. A method as in claim 13, where the device is used in the treatment of atrial fibrillation

16. A method as in claim 13, where the device is used to treat movement disorders.

17. A method as in claim 13, where the device is used to treat depression

18. A method of treating neurologic disorders comprising the steps of identifying the target tissue responsible for the disorder; placing a mechanical stress device proximate to the spinal nerve bundle; the whereby said mechanical stress device remains proximate to the spinal nerve bundle; whereby said mechanical stress device affects the electrical activity of the appropriate nerve pathway connecting the cortical surface and the target tissue; whereby the affected electrical activity of the nerve pathway mitigates the neurologic disorder.

19. A method as in claim 18, where the device is used in the treatment of atrial fibrillation

20. A method as in claim 18, where the device is used to treat movement disorders.

21. A method as in claim 18, where the device is used to treat depression

22. A method as in claim 15, where the device is directly coupled to an electrical generator.
